(19) United States
(12) Patent Application Publication
(10) Pub. No.: US 2026/0179288 A1
(43) Pub. Date: Jun. 25, 2026

(54) RECONSTRUCTION OF AN IMAGE IN MEDICAL IMAGING

(71) Applicant: Siemens Healthineers AG, Forchheim (DE)

(72) Inventors: Jessica Schulz, Forchheim (DE); Michael Manhart, Fürth (DE); Daniel Punzet, Magdeburg (DE); Robert Frysch, Magdeburg (DE); Georg Rose, Magdeburg (DE)

(21) Appl. No.: 19/426,856

(22) Filed: Dec. 19, 2025

(30) Foreign Application Priority Data
Dec. 23, 2024 (DE) 10 2024 212 291.2

(51) Int. Cl. G06T 12/00; G06T 7/00; G06T 7/33

(52) U.S. Cl. CPC G06T 12/00; G06T 7/0012; G06T 7/344; G06T 2200/04; G06T 2207/10081; G06T 2207/30004; G06T 2207/30168; G06T 2210/41; G06T 2211/40

(57) ABSTRACT

A method for reconstructing an image of an object in medical imaging includes: providing a first truncated projection image of the object that represents a region to be mapped of the object projected from a first projection source position into a first projection plane; providing a three-dimensional (3D) model specific to the object, including a modeled region to be mapped which corresponds to the region to be mapped of the object; generating a first supplementary forward projection of a first supplementation region of the 3D model, which extends beyond and adjoins the modeled region to be mapped, from the first projection source position into the first projection plane; and reconstructing the image based on the first truncated projection image and the first supplementary forward projection of the first supplementation region.

The present patent document claims the benefit of German Patent Application No. 10 2024 212 291.2, filed Dec. 23, 2024, which is hereby incorporated by reference in its entirety.

### TECHNICAL FIELD

The present disclosure relates to a method for reconstructing an image of an object in medical imaging. The present disclosure further relates to a computed tomography method, an imaging modality, and a computer program.

### BACKGROUND

If tomographic images from projections are to be unambiguously reconstructed, the projection data fully maps the entire cross-section (in the lateral direction) of the object or patient. If the projections are not complete but instead are cut off, the projections are known as truncated. Truncated projection data occurs in medical imaging, for example, if the patient is too large for the detector or the projections were deliberately captured only in part (e.g., by collimation of the beam path), for example, for dose minimization (volume-of-interest (VOI) imaging).

In order to obtain usable reconstruction results, the truncated projections are expanded (extrapolated) using well-established methods, e.g., by a heuristic extrapolation model, in order to minimize the occurrence of image artifacts. Such approaches are described in the following two articles: Sourbelle et al., “Reconstruction from truncated projections in CT using adaptive detruncation,” Eur Radiol. 15, 1008-1014 (2005). https://doi.org/10.1007/s00330-004-2621-9 and Hsieh et al., “A novel reconstruction algorithm to extend the CT scan field-of-view,” Medical Physics 31 (9), 2004, pp. 2385-2391. DOI: 10.1118/1.1776673.

However, an existing volume data set (e.g., a planning CT) of the patient may already be available. This may be used for a further imaging method, for example, in order to reduce radiation exposure. This means that newly obtained 2D projection data has to be registered on the already available 3D volume data set.

The aim of placing 2D projection data and 3D volume data of an object in registration with one another is to establish a suitable geometric transformation that sets the two data sets in a common spatial relationship to one another. An article in the state of the art provides an overview of this matter (see, e.g., van de Kraats et al., “Standardized evaluation methodology for 2-D-3-D registration,” IEEE Transactions on Medical Imaging, vol. 24, no. 9, pp. 1177-1189 September 2005, doi: 10.1109/TMI.2005.853240).

Two-dimensional (2D)-three-dimensional (3D) registration of projection and volume data involves 2D projections of a certain region of the object (e.g., the entire object) and a 3D volume data set of the same object or patient. The two data sets are conventionally transformed into a common spatial frame of reference by repeatedly simulating forward projections of the volume data set (“digitally reconstructed radiographs” (DRRs)). The parameters of the geometric transformation under which these DRRs exhibit the least error compared to the given projections in terms of a similarity metric are established by optimization. Applying a geometric transformation using the parameters calculated in this way to the given data then carries out the established registration.

This approach becomes difficult in the case of truncated projection data (i.e., the object has only been partially mapped), since it may then only be possible to use feature-based similarity metrics for comparing DRRs and existing projection data.

### SUMMARY AND DESCRIPTION

The object of the present disclosure is accordingly to improve the quality of the reconstruction of images in medical imaging.

The scope of the present disclosure is defined solely by the appended claims and is not affected to any degree by the statements within this summary. The present embodiments may obviate one or more of the drawbacks or limitations in the related art.

The disclosure accordingly provides a method for reconstructing an image (e.g., a tomographic image or 3D image) of an object in medical imaging. The object may be a patient, part of a patient, or an object in a patient. Such an object might be, for example, a catheter or an implant. The medical imaging may be carried out by computed tomography, angiography, and the like. The respective medical imaging methods are based on projections or transilluminations of the object. At least one image is obtained or reconstructed by the medical imaging. It is established from the raw data from the imaging. The image may be a tomographic image.

In one act, the method includes providing a first truncated projection image of the object that represents the region to be mapped of the object projected from a first projection source position in a first projection plane. The truncated projection image does not represent the object completely but rather only in part. It is therefore cut off, i.e., truncated. The truncated projection image only reproduces the region to be mapped of the object (e.g., volume of interest (VOI)). The projection rays extend from the first projection source position through the object to the first projection plane.

The truncated projection image may be provided in many ways. For example, the truncated projection image may be obtained and made available in a memory device. The truncated projection image is thus captured and, for example, made available digitally. Alternatively, the truncated image may also be received, for example, via a data network (e.g., the internet) and provided in this manner. As a further alternative, the truncated projection image may be provided by being read out from a memory. Such a memory may be a hard disk or a portable memory unit.

In another act, the method involves providing a 3D model of the object specific to the object, including a modeled region to be mapped that corresponds to the region to be mapped of the object. The 3D model may be provided in the same manner as the provision of the truncated projection image that has just been presented, e.g., by capturing, receiving, reading out, and so on. The 3D model specific to the object is a volume data set that spatially represents the object. The volume data set and thus the 3D model are obtained from captures of the object that have previously been obtained. The 3D model thus shows the same or similar details as the object itself. The 3D model also contains a region that corresponds to the real region of the object which is mapped by the truncated projection image. The corresponding region in the 3D model is denoted “modeled region to be mapped.” This may be the modeled VOI. There may be (e.g., small) differences between the real object and the model. For example, deformations occur in a patient, for example, due to breathing, etc. Depending on how far apart a current projection image and previously obtained volume data set are in temporal terms, small modifications are moreover to be expected (e.g., a tumor has formed, an organ has changed, etc.).

According to a further act of the method, a first supplementary forward projection of a first supplementation region, which extends beyond and adjoins the modeled region to be mapped, of the 3D model is generated from a first projection source position into the first projection plane. A first supplementary forward projection, i.e., a modeled projection image, starting from the first projection source position is thus calculated on the basis of the 3D model. This means that a projection is simulated with the assistance of the 3D model specific to the object. This simulation or supplementary forward projection does not relate or not primarily to the region to be mapped of the object, but instead to a first supplementation region. This supplementation region may include part of the region to be mapped but extends beyond the part of the region to be mapped. In any event, the first supplementation region adjoins the region to be mapped without there being any gap therebetween. The aim is namely to avoid artifacts that arise at the boundaries of regions to be mapped.

A further act of the method involves reconstructing the image on the basis of the truncated projection image and the first supplementary forward projection of the first supplementation region. Reconstruction is carried out, for example, with the assistance of a reconstruction algorithm into which the truncated projection image and the first supplementary forward projection of the first supplementation region are input. The reconstruction algorithm may also use further (e.g., truncated) projection images and further supplementary forward projections for reconstruction. A set of a plurality of such projection images and supplementary forward projections may be used for the reconstruction.

The first supplementary forward projection or further supplementary forward projections advantageously supply additional data for reconstructing the image, such that the quality of the reconstruction may be improved. The additional data of the supplementary forward projection or forward projections moreover originate from an object-specific model of the object to be mapped and are not based on a purely heuristic basis. Artifacts may be reduced in this way too. Since the model used here is a previously obtained object-specific volume data set, the method may be understood as both an extrapolation and a registration method.

One exemplary embodiment provides that the first truncated projection image and the first supplementary forward projection of the supplementation region are registered relative to one another. The first truncated projection image and the first supplementary forward projection originate from two different independent methods. The truncated projection image is generated by a real projection through the real object. The supplementary forward projection, by contrast, is simulated by an appropriate projection algorithm. These different image generation processes may require the images obtained to be registered relative to one another. This means that the two images, namely the truncated projection image and the supplementary forward projection, have to be oriented or adapted relative to one another. This registration may involve translation, rotation, magnification, contrast, or the like. In one example, the translation and rotation of the object may be optimized. Scaling, contrast, etc. may also be possible. Scaling may be known from the given metadata of the volume data set and thus need not necessarily be selected via optimization. Once such registration is complete, the two images may be brought into line with one another as well as possible and are capable of optimally supplementing one another.

In a further embodiment, the method additionally includes generation, from the first projection source position into the first projection plane, of a second supplementary forward projection of a second supplementation region of the 3D model, which extends beyond and adjoins the modeled region to be mapped. The image is then also reconstructed on the basis of the second supplementary forward projection of the second supplementation region. A second supplementary forward projection is thus obtained here for which there may be no corresponding projection image, similarly to how there is likewise no projection image for the first supplementary forward projection. The second supplementation region may adjoin a side of the modeled region to be mapped that is opposite to the first supplementation region. This means that the modeled region to be mapped is located precisely between the first supplementation region and the second supplementation region of the 3D model. The regions directly adjoin one another in pairs. Accordingly, a corresponding series of projections directly adjoining one another is obtained: first supplementary forward projection, first truncated projection image, and second supplementary forward projection. The desired image is thus additionally reconstructed on a pair of supplementary forward projections that are located, for example, on the left-hand and right-hand side of the actual truncated projection image. Artifacts that arise due to the two side edges of the truncated projection image may thus be avoided to some extent.

The forward projections of the model may be simulated simultaneously on both sides. The model may be placed with a certain translation and rotation and then the two (left- and right-hand side) supplementary forward projections are simultaneously “captured.” Simulation is carried out with a large virtual detector that would be large enough to capture the left-hand, central, and right-hand regions. Since the projection in the middle is given, it is not co-simulated for computing time reasons. The two supplementary (left-hand and right-hand) forward projections may theoretically also be simulated in succession. However, since the two supplementary forward projections (or “supplementation projections”) are intended to represent the model in the same translation/rotation (so that consistent projections are obtained), this may not make sense.

A further embodiment provides that the two supplementary forward projections correspond to a first pair that is generated in relation to the first projection source position. Further, adjoining a second truncated projection image, a second pair of supplementary forward projections is obtained in a similar manner in a second projection plane that differs from the first by way of the 3D model. The second pair is generated in relation to a second projection source position that differs from the first. Additionally, the image is reconstructed on the basis of both pairs of supplementary forward projections and both truncated projection images. This means that the image may be reconstructed not only on the basis of one pair of forward projection images, but rather on at least one further pair of supplementary forward projections or forward projection images. Each pair of supplementary forward projections is in each case generated from or obtained for a different projection source position. This means that the two truncated projection images are obtained from two different projection source positions, i.e., the respective sources of the projection are located in different positions. The same source position as for the associated truncated projection image is used when simulating each pair of supplementary forward projections. In this way, further triplets of forward projection pairs including the associated projection image may also be provided for the reconstruction. Reconstruction quality may thus be further improved.

In a further embodiment, a first intermediate function is created for the first truncated projection image and the first supplementary forward projection (which together form a first doublet). Additionally, a second intermediate function is created for the second truncated projection image and an associated supplementary forward projection (which together form a second doublet). Further, an error (i.e., any desired error metric) between the two intermediate functions is calculated and optimized. The first truncated projection image and the first supplementary forward projection may thus be brought into line with one another. A first intermediate function arising from a corresponding linkage between projection image and supplementary forward projection is used for this purpose. The intermediate function may be formed on the basis of the projection parameters underlying the first truncated projection image and the first supplementary forward projection. Once registration is successful, the truncation is completely eliminated. The size of the supplementary forward projections may be selected such that the true registered volume may be completely mapped.

According to an analogous embodiment, a first intermediate function is created for the first truncated projection image and the first pair of supplementary forward projections. Also, a second intermediate function is created for the second truncated projection image and the second pair of supplementary forward projections. Further, an error (i.e., any desired error metric) between the two intermediate functions is calculated and optimized. A respective intermediate function is thus created for the first triplet (first projection image and first pair of supplementary forward projections) and for the second triplet (second projection image and second pair of supplementary forward projections) and an error between them is minimized. The supplementary forward projections are thus placed in registration with their corresponding projection images. Reconstruction quality is in turn increased by these triplets that are themselves in registration.

A further developed embodiment provides that epipolar lines in the supplementary forward projections are used to calculate the respective intermediate function, wherein the epipolar lines together with the two projection source positions are located in one plane. This means that the epipolar lines of the first doublet (first projection image and first supplementary forward projection) and the second doublet (second projection image and associated supplementary forward projection) or the epipolar lines of the first triplet (first projection image and first pair of supplementary forward projections) and of the second triplet (second projection image and second pair of supplementary forward projections) together with the associated projection source positions define a plane. As soon as the error of two intermediate functions relating to corresponding epipolar lines is minimal, all the projection images and supplementary forward projections are optimally in registration with one another.

The epipolar lines are defined purely by the geometry of the two projections and two source positions. They are thus section lines of the fan of planes about the baseline connecting the source positions with the respective (virtual) detectors.

A value of the respective intermediate function may be calculated for each of these epipolar lines. On the basis of two intermediate functions, an error function may now be formulated that indicates how much two intermediate functions differ. If the error between the intermediate function associated with projection source position Ci and the intermediate function associated with projection source position Cj is minimal, it may be assumed that the projections from the two positions are consistent with one another.

The error function only indirectly indicates whether the respective supplementary forward projection matches the projection image. It primarily indicates whether the one projection relating to Ci matches or is consistent with the other projection relating to Cj. If that is the case, it may be concluded that the projection image and supplementary forward projection are also consistent with one another.

Optimization takes place in the volume region (3D). If just one single projection triplet or doublet (2D) “matches,” it does not yet mean that the volume is correctly oriented in 3D space. This requires another projection triplet or doublet. The error function (as the difference between two intermediate functions) may indicate whether the two pairs of projection triplets or doublets ultimately “match”.

A further embodiment provides that values of the respective intermediate function are calculated for the respective supplementary forward projections before the respective projection images of the object are provided. In particular, the values of the intermediate functions may be calculated in advance. Calculating the error or error function only makes sense once the truncated projection data is available. The supplementary forward projections are consistent with one another because they are generated in that way. For example, an object is positioned in some manner in the 3D volume space and the forward projections are simulated in the supplementation region. These outer regions may thus be consistent with one another by design. However, the aim is to select the supplementation region to be consistent with the (given) truncated projection image (inner region) and the projections of the supplementation region are optimized accordingly. To this end, the object is differently positioned in volume space and again forward projected etc.

A still further exemplary embodiment provides that the 3D model is based on captures, in particular planning scans, of the object or of a further object of the same type as the object. Planning scans may be of higher quality than subsequent scans because they are used for diagnostic purposes and may originate from a CT scanner. The truncated data to be extended, on the other hand, may be “interventional” scans and may be captured, for example, by C-arm CT (CBCT). At least with regard to contrast resolution, the image quality of C-arm CTs may be poorer than that of conventional CTs. The planning scans may also originate from a prior C-arm CT scan and even truncated CT projections may benefit from the present disclosure. The corresponding planning data set or “prior” is used here to enlarge the scope of the overall data obtained such that elevated image quality of the reconstructed images may be achieved with comparatively low radiation exposure.

In an embodiment, the image to be reconstructed is a tomographic image or a 3D image. This means that a cross-sectional image or a 3D model of the object is obtained from the available data. The quality of the 3D model per se or of the cross-sectional image is improved on the basis of supplementary forward projections that may be provided using the 3D model of the object.

The above-stated object may also be achieved by a computed tomography method in which images are reconstructed according to the above-described method. The computed tomography method may be a CT method, a CBCT (cone-beam computed tomography) method, an angiography method, or the like. Reconstruction quality may in any event be improved by the above method.

The above object is moreover also achieved by an imaging modality with a data processing device and a control device that are configured to carry out an above-described method. The data processing device may have one or more processors and one or more memory devices and optionally any interfaces. The control device may in turn have control elements for imaging components, as well as one or more processors, one or more memory devices and one or more interfaces. The advantages and developments of the method set out above apply mutatis mutandis to the imaging modality as described herein. In this case, the stated method acts are corresponding functional features of sub-elements of the imaging modality.

A computer program is furthermore provided including commands that, on execution of the program by the stated imaging modality, cause the imaging modality to carry out the method set out above. A non-transitory computer-readable medium including the computer program having such commands may similarly be provided.

The present disclosure is now explained in greater detail with reference to the appended drawings.

### BRIEF DESCRIPTION OF THE DRAWINGS

FIG. 1 depicts a schematic view of an example of a CT system.

FIG. 2 depicts a schematic view of an example of two projections.

FIG. 3 depicts an example of supplementary simulated forward projections of regions of an extrapolation model not included in the projection data.

FIG. 4 depicts an example of the calculation of intermediate function values along straight lines on a virtual detector of the assembled projection data for pairs of projections.

FIG. 5 depicts a schematic flowchart for an embodiment of a method.

### DETAILED DESCRIPTION

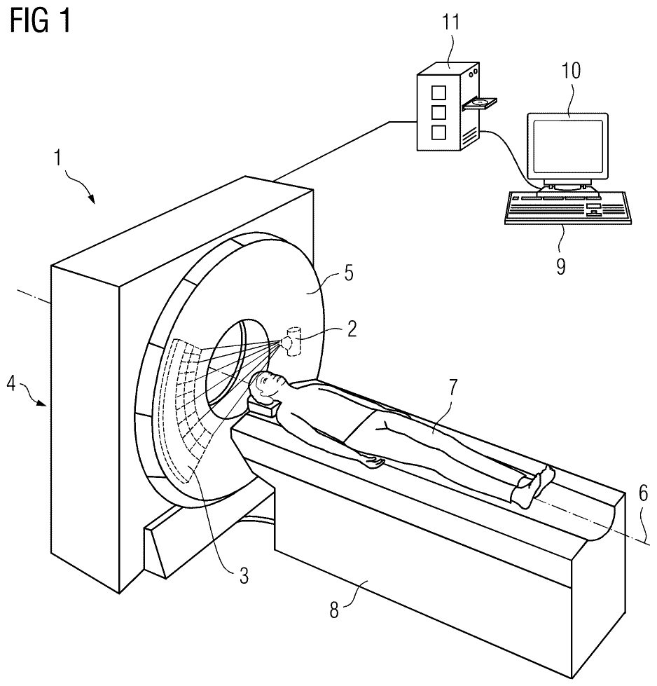

FIG. 1 shows a schematic representation of an advantageous embodiment of a proposed X-ray machine as an imaging modality or medical CT scanner (computed tomography scanner) 1. The CT scanner 1 may include an X-ray source 2, an X-ray detector 3, and a processing unit 11 (e.g., one or more processors) as a type of signal processing unit. The X-ray source 2 and the X-ray detector 3 are arranged opposite one another. The X-ray source 2 may be configured such that the X-ray source irradiates the X-ray detector 3 with X-rays along an X-ray incidence direction.

The CT apparatus 1 may also include a gantry 4 with a rotor 5. The X-ray source 2 and the X-ray detector 3 may be arranged in a defined arrangement on the rotor 5, in particular integrated into the rotor 5 or attached to the rotor 5. The rotor 5 may be rotatably mounted about an axis of rotation 6. The object under examination 7 to be mapped may lie on a patient positioning device 8 and be moved along the axis of rotation 6 through the gantry 4. The processing unit 11 may be used to control the CT scanner 1 and to calculate cross-sectional images or volume images of the object under examination 7. An input device 9, for example a keyboard, and an output device 10, for example a screen and/or a display, may be connected, (e.g., coupled for signaling), to the processing unit 11. The input device 9 may advantageously be integrated into the output device 10, for example, in the case of an input display, in particular a resistive and/or capacitive input display. The output device 10 may be configured to display a graphical representation of the X-ray image data set or any modeled images.

The schematic representations included in the described figures reproduce neither scale nor proportions.

A further example (not shown in the figures) may relate to a mono-planar X-ray system with a C-arm that is held on a base in the form of a, for example six-axis, industrial or articulated robot, on the ends of which are attached an X-ray source, for example, an X-ray source with X-ray tube and collimator, and an X-ray image detector as image capture unit. The embodiment of the diagnostic X-ray machine is not dependent on the industrial robot. Conventional (stationary or mobile) C-arm machines may also be used.

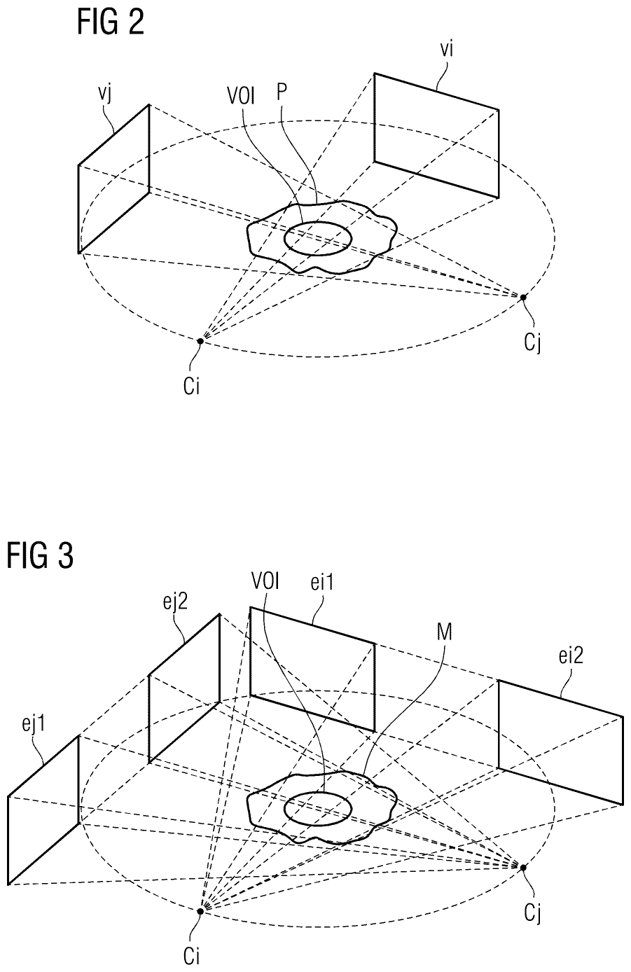

FIG. 2 is a schematic diagram of the performance of forward projections vi and vj. Forward projection vi is obtained by projection of a three-dimensional object P having a volume region of interest VOI. Projection is carried out from a projection position Ci into a first projection plane. Forward projection vj is obtained in similar manner by projection of the three-dimensional object P from the projection position Cj into a second projection plane that differs from the first. Projection positions Ci and Cj may be located, for example, on a circular path, spiral path, or a path modulated in a more complex manner. The 3D data of the object P may be registered on the 2D projection data by comparing forward projections vi and vj with actual projections (2D data).

The present disclosure views the problem of registration from a new angle: for example, instead of calculating simulated forward projections in the region of the object P for which projections are given, regions for which there is no projection data are forward projected in simulated form according to FIG. 3, so yielding simulated supplementary forward projections ei1, ei2, ej1, ej2.

An existing volume data set (e.g., a planning CT) of the patient may already be available. By registering this “prior” on the projection data to be reconstructed, a more accurate, patient-specific extrapolation and thus a better reduction of image artifacts would be possible. To this end, 2D projection data have to be registered on 3D volume data.

An extrapolation model M is used for the supplementary forward projection. This extrapolation model M may be identical to or supplement the object model P originating from a prior data set. The extrapolation model M may forward project a first supplementation region and a second supplementation region into the first projection plane from the projection position Ci, so giving rise to modeled projection images (also denoted supplementary forward projections ei1 and ei2 for short). The two supplementary forward projections ei1 and ei2 based on the supplementation regions are located, for example, to the left and right of a truncated projection image (not shown in FIG. 3). A first supplementation region and a second supplementation region may likewise be forward projected from projection position Cj into the second projection plane in the same way in order to obtain the respective supplementary forward projections ej1 and ej2. These two supplementary forward projections ej1 and ej2 are also located, for example, to the left and right of a truncated projection image that was captured from projection position Cj (also not shown in FIG. 3).

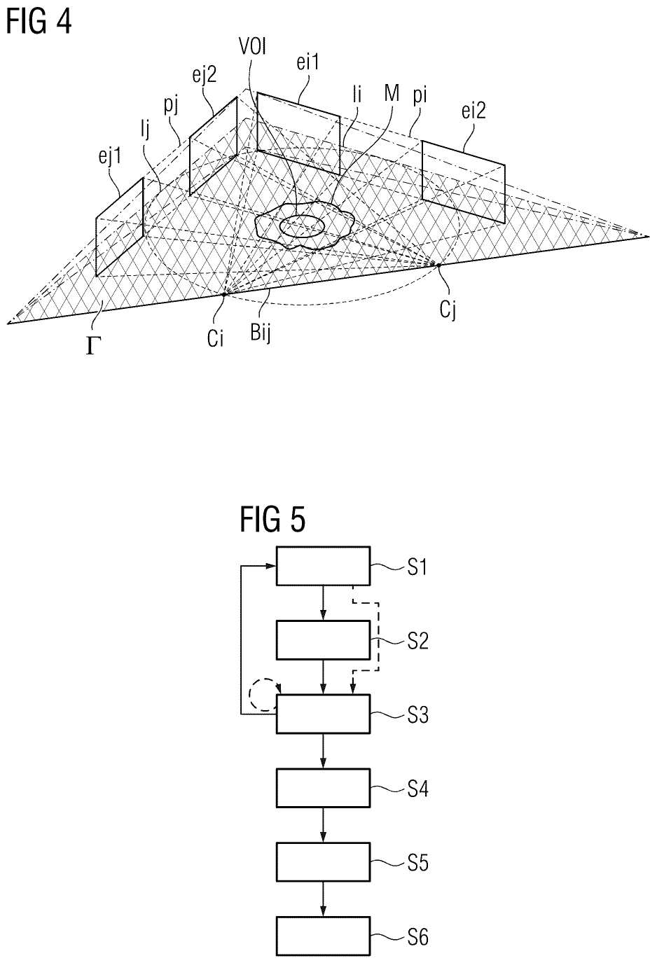

As FIG. 4 shows, an actual or truncated projection image pi that was captured from projection position Ci may be supplemented by or combined with the two supplementary forward projections ei1 and ei2 in the first projection plane. The supplementary forward projections ei1 and ei2 may be configured or arranged in such a way that they directly adjoin projection image pi on opposite sides thereof. It is in principle possible to supplement truncated projection image pi on just one side, in particular if truncation is on just one side. Supplementation on both sides is, however, advantageous when truncation is on both sides. If reconstruction is to be accurate, all the projections of the object may be completely untruncated. The more truncation there is, the more severe are the image artifacts arising from the reconstruction of truncated data.

The size of the supplementary forward projections ei1 and ei2 may be identical to the size of the truncated projection image pi. Alternatively, the supplementary forward projections ei1 and ei2 may, however, also be larger or smaller than the truncated projection image pi. They may be of sufficient size for the object in pi+ei1+ei2 to be completely projected. Since, is may not precisely be known “how much of the object is missing,” the supplementary forward projections used will tend to be selected larger.

Similarly, a projection image pj obtained from projection position Cj may also be supplemented by one or more supplementary forward projections ej1 and ej2 in the second projection plane. A projection data set from which reconstruction is performed may have around 500 projections. The described methodology with the intermediate functions etc. does not, however, have to be carried out for all 500 projections. It is sufficient to compare a sufficient number of projection pairs. In theory, two projections (i.e., one projection pair as depicted) are sufficient by themselves for carrying out registration. Registration indicates how the model has to be placed in volume space in order to obtain forward projections consistent with the truncated data. Once these (e.g., translation and rotation) parameters for the volume data set have been obtained, the volume may be projected from further directions (e.g., from all 500 projection directions of the truncated scan), resulting in a complete extrapolated projection data set with which reconstruction may be performed.

Epipolar lines li and lj may be used to orient projection images pi and pj together with their supplementary forward projections ei1 and ei2, ej1 and ej2 relative to one another. These epipolar lines li and lj are located in a common plane Γ with a baseline Bij that extends through projection positions Ci and Cj. In particular, the values of an intermediate function may be mutually independently calculated for regions ei1, pi, and ei2. By optimization, as described below, the respective registration parameters for positioning the model may be optimized to the effect that the intermediate function values correspond to one another on corresponding epipolar lines. The same may be carried out for regions ej1, pj, and ej2 in relation to epipolar line lj. As a result of these optimizations, the supplementary forward projections with their respective projection images are optimally oriented relative to one another. This means that the data is mutually consistent or the virtual supplementary forward projections are optimally registered with the respective projection images.

In detail, simulated supplementary forward projections ei1, ei2, ej1, ej2 are combined with the known projection data pi, pj of the remainder of the object region. In this case, the sought parameters of the geometric transformation are not calculated by optimization of a similarity metric (the DRRs no longer approximate the given projection data but rather extend it). Instead, data consistency conditions to be met for “correct” or consistent projection data are used to form an error metric that is minimized by optimization. These consistency conditions may be calculated in the form of an intermediate function from pairs of projections from different source positions (see FIG. 4). The registration of the data sets is thus carried out only indirectly as exactly opposite regions of the object are given and are forward projected. Registration is achieved indirectly by calculating consistent extrapolation and may thus be considered a kind of inverted DRR registration method. That is, instead of approximating the known projection data by forward projection of a volume data set in the region of the known projection data, the known projection data is extrapolated in the exactly opposite region by forward projections of a volume data set.

The intermediate function may be calculated according to Punzet et al., “Extrapolation of Truncated C-arm CT Data using Grangeat-based Consistency Measures,” Proceedings of the CT Meeting 2018, The Fifth International Conference on Image Formation in X-ray Computed Tomography. Salt Lake City, UT, USA, 2018. With regard to the following equations, explicit reference is made to this publication where the equations are explained in greater detail. The intermediate function is obtained from Grangeat's fundamental relation as

$${S}_{\text{?}} ( \hat{n} ) := - {\int }_{{S}^{2}} d {m}^{\text{?}} {δ}^{′} ( {m}^{\text{?}} \cdot {n}^{\text{?}} ) {g}_{\text{?}} ( \hat{m} ) = \frac{\partial }{\partial ξ} Γ ( {n}^{\text{?}} , ξ ) {\vert}_{ξ \text{?}} .$$

$$\text{?} \text{indicates text missing or illegible when filed}$$

It thus establishes a relationship between the X-ray transform (the projection data)

$$𝒳 ( {C}_{\text{?}} , {m}^{\text{?}} ) := {\int }_{0}^{∞} f ( {C}_{\text{?}} + {m}^{\text{?}} λ ) d λ := {g}_{i} ( {m}^{\text{?}} )$$

$$\text{?} \text{indicates text missing or illegible when filed}$$

and the 3D radon transform (plane integrals in volume space):

$${ℛ}_{3} ( {n}^{\text{?}} , ξ ) := {\int \int \int }_{{ℝ}^{3}} f ( r ) δ ( r \cdot {n}^{\text{?}} - ξ ) dr := Γ ( {n}^{\text{?}} , ξ )$$

$$\text{?} \text{indicates text missing or illegible when filed}$$

This enables calculation of the values of the intermediate function either via calculation of the plane integral and subsequent derivation in volume space or via calculation of line integrals in the projection data (i.e., on the detector) together with derivation in the perpendicular direction. Calculating the intermediate function via the plane integrals in volume space may not be possible in practice. The intermediate function values may alternatively be calculated from the projections.

By using epipolar geometry, it is possible to identify epipolar lines in pairs of projections from the same acquisition (e.g., for CBCT scans conventionally a circular trajectory over around) 200° and use them for calculating the intermediate function. Since the intermediate functions or their values that are calculated from the two projections relate to the same plane integral in volume space, the intermediate functions from the two projections may correspond. A consistency condition for pairs of projections that are consistent with one another is thus obtained: the greater the difference in the intermediate functions of two projections, the more inconsistent are the two projections to one another. The deviation of two such intermediate functions from one another means that there are certain differences (e.g., geometric differences, artifacts, noise, etc.) between the two projections.

This consistency metric is used for the described method in order to identify an extrapolation of the limited data that is consistent for a number of pairs of projections. Epipolar lines li, lj may be identified in the projection data composed of given projections pi, pj and forward projected DRRs ei1, ei2, ej1, ej2 and intermediate functions calculated (see FIG. 4).

The article by Punzet et al. already makes use of the described consistency metric to optimize the conventionally used heuristic extrapolations with regard to consistent projections, for example, with an ellipsoid. The crucial difference of the method described here resides in making use of the consistency conditions in order, instead of simple extrapolation models, to use an existing volume data set (e.g., prior CT or prior CBCT) not only for extrapolation but rather simultaneously also for registration on the truncated projection data set. Here, a crucial factor for rapid calculation of the optimal extrapolation is the possibility of calculating the values of the intermediate functions of the extrapolation DRRs ei1, ei2, ej1, ej2 in advance (e.g., as soon as the prior data set is available). In this way, the space of the optimization parameters has already been sampled in advance. At the time of the actual reconstruction, all that remains is to optimally combine the intermediate function values of the respective three subregions. Subsequent local optimization may be used to refine the result.

FIG. 5 shows an exemplary embodiment of a method in a flowchart. In act S1, a first truncated projection image of the object is provided (e.g., in practice a pair of projection images may be required for adequate consistency) that represents a region to be mapped of the object in a projection plane. Provision of the projection image may mean that it is obtained, captured, received, read out, or the like. The projection image is truncated and thus cut off on at least one side.

In act S2, a 3D model of the object specific to the object, including a modeled region to be mapped which corresponds to the region to be mapped of the object, is provided. The 3D model is thus likewise provided by being obtained, captured, received, read out, or the like. The 3D model may be obtained beforehand with the assistance of planning scans.

In act S3, a first supplementary forward projection or a supplementary forward projection pair of a first supplementation region, which extends beyond and adjoins the modeled region to be mapped, is generated using the 3D model. At least one supplementary forward projection is thus obtained, not with the assistance of a heuristic 3D model but rather with the assistance of a 3D model specific to the object.

Acts S1 and S3 may be repeated for a plurality of truncated projection images. Act S3 taken alone may likewise be repeated in order to forward project a plurality of supplementation regions for a single truncated projection image.

The intermediate function (e.g., pairs) may be calculated and the error minimized via optimization of the parameters of the volume data set in a subsequent optional act S4. In act S5, the entire supplementation regions with optimum registration parameters (e.g., from the optimization) may then be projected.

In act S6, the image is reconstructed on the basis of the truncated projection image and the first supplementary forward projection of the first supplementation region. The sought image is thus reconstructed with the assistance of data that has been supplemented with the assistance of an object-specific model.

The field of application of the present disclosure resides in the described scenario in which, for reasons of dose reduction or limited detector size, only truncated projection data is available for image reconstruction. In particular, in radiotherapy or in interventional procedures, planning scans in the form of a CT prior may be available. Extrapolation with this prior may provide better extrapolation than simple heuristic methods. Correct registration of the data sets is required for this extrapolation or may be understood as a secondary product of the disclosure. The ultimate benefit is improved image quality of the reconstructed images and a reduced dose for the patient.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for reconstructing an image of an object in medical imaging, the method comprising:
providing a first truncated projection image of the object that represents a region to be mapped of the object projected from a first projection source position into a first projection plane;
providing a three-dimensional (3D) model specific to the object, wherein the 3D model comprises a modeled region to be mapped that corresponds to the region to be mapped of the object;
generating a first supplementary forward projection of a first supplementation region of the 3D model, which extends beyond and adjoins the modeled region to be mapped, from the first projection source position into the first projection plane; and
reconstructing the image based on the first truncated projection image and the first supplementary forward projection of the first supplementation region.

2. The method of claim 1, wherein the first truncated projection image and the first supplementary forward projection are registered relative to one another.

3. The method of claim 2, further comprising:
generating a second supplementary forward projection of a second supplementation region of the 3D model, which extends beyond and adjoins the modeled region to be mapped on a side of the modeled region to be mapped that is opposite to the first supplementation region, from the first projection source position into the first projection plane,
wherein the reconstructing of the image is also based on the second supplementary forward projection of the second supplementation region.

4. The method of claim 3, wherein the first supplementary forward projection and the second supplementary forward projection correspond to a first pair of supplementary forward projections that is generated in relation to the first projection source position,
wherein, adjoining a second truncated projection image, a second pair of supplementary forward projections is obtained in a similar manner in a second projection plane that differs from the first projection plane by way of the 3D model,
wherein the second pair of supplementary forward projections is generated in relation to a second projection source position that differs from the first projection source position, and
wherein the image is reconstructed based on both pairs of supplementary forward projections and both truncated projection images.

5. The method of claim 4, wherein a first intermediate function is created for the first truncated projection image and the first supplementary forward projection,
wherein a second intermediate function is created for the second truncated projection image and an associated supplementary forward projection, and
wherein an error between the first intermediate function and the second intermediate function is calculated and optimized.

6. The method of claim 5, wherein epipolar lines in the supplementary forward projections are used to calculate the respective intermediate function, and
wherein the epipolar lines together with the first projection source position and the second projection source position are located in one plane.

7. The method of claim 5, wherein values of the respective intermediate function for the respective supplementary forward projections are calculated before the respective projection images of the object are provided.

8. The method of claim 4, wherein a first intermediate function is created for the first truncated projection image and the first pair of supplementary forward projections,
wherein a second intermediate function is created for the second truncated projection image and the second pair of supplementary forward projections, and
wherein an error between the first intermediate function and the second intermediate function is calculated and optimized.

9. The method of claim 8, wherein epipolar lines in the supplementary forward projections are used to calculate the respective intermediate function, and
wherein the epipolar lines together with the first projection source position and the second projection source position are located in one plane.

10. The method of claim 8, wherein values of the respective intermediate function for the respective supplementary forward projections are calculated before the respective projection images of the object are provided.

11. The method of claim 1, wherein the 3D model is based on previously obtained captures of the object or of a further object of a same type as the object.

12. The method of claim 1, wherein the image to be reconstructed is a tomographic image or a 3D image.

13. The method of claim 1, wherein the medical imaging is performed by a computed tomography scanner.

14. An imaging modality comprising:
a data processing device; and
a control device,
wherein the data processing device and control device are configured to:
provide a first truncated projection image of an object that represents a region to be mapped of the object projected from a first projection source position into a first projection plane;
provide a three-dimensional (3D) model specific to the object, wherein the 3D model comprises a modeled region to be mapped that corresponds to the region to be mapped of the object;
generate a first supplementary forward projection of a first supplementation region of the 3D model, which extends beyond and adjoins the modeled region to be mapped, from the first projection source position into the first projection plane; and
reconstruct the image based on the first truncated projection image and the first supplementary forward projection of the first supplementation region.

15. The imaging modality of claim 14, wherein the imaging modality is a computed tomography scanner.

16. A non-transitory computer-readable medium comprising a computer program having commands that, on execution of the computer program by an imaging modality, cause the imaging modality to:
provide a first truncated projection image of an object that represents a region to be mapped of the object projected from a first projection source position into a first projection plane;
provide a three-dimensional (3D) model specific to the object, wherein the 3D model comprises a modeled region to be mapped that corresponds to the region to be mapped of the object;
generate a first supplementary forward projection of a first supplementation region of the 3D model, which extends beyond and adjoins the modeled region to be mapped, from the first projection source position into the first projection plane; and
reconstruct the image based on the first truncated projection image and the first supplementary forward projection of the first supplementation region.
